(19) United States
(12) Patent Application Publication
(10) Pub. No.: US 2005/0244859 A1
(43) Pub. Date: Nov. 3, 2005

(54) Cell based assay

(75) Inventors: Zhong Zhong, Bridgewater, NJ (US); Glen L. Kelley, Freehold, NJ (US); Thomas J. Mercolino, Stockton, NJ (US); Robert Zivin, Skillman, NJ (US); John J. Siekierka, Towaco, NJ (US)

Correspondence Address: WOODCOCK WASHBURN LLP, ONE LIBERTY PLACE, 46TH FLOOR, PHILADELPHIA, PA, 19103, US

(73) Assignee: Ortho McNeil Pharmaceutical, Inc.

(21) Appl. No.: 11/081,049

(22) Filed: Mar. 14, 2005

Related U.S. Application Data
(63) Continuation of application No. 09/663,306, filed on Sep. 15, 2000, now abandoned.
(60) Provisional application No. 60/155,353, filed on Sep. 22, 1999.

(51) Int. Cl. C12Q 1/68; C07H 21/04; C12P 21/06; C12N 9/22; C12N 15/09

(52) U.S. Cl. 435/6; 435/69.1; 435/199; 435/320.1; 435/325; 536/23.2

(57) ABSTRACT

The present invention provides a whole-cell biological assay that measures changes of endogenous genes under control of an exogenously introduced transcription factor. The exogenous transcription factors of the present invention may be designed such that each is activated by specific extracellular ligands. Therefore cells containing exogenous transcription factors of the present invention provide a generic means to which many extracellular ligands may be tested without undue adaptation to the assay.

### BACKGROUND OF THE INVENTION

Extracellular ligands interact with specific cellular receptors and modulate the receptor. Examples of these modulations include oligomerization, activation of endogenous enzymatic activity, exposure of binding sites for accessory proteins, or exposure of endogenous enzyme substrate sites. Receptor modulation transduces a signal in the cell through pathways including phosphorylation cascades, ion concentration changes, phosphatidylinositol metabolism, cyclic AMP production, and guanidinyl nucleotide transfer. The intracellular pathways can receive transduced signals from multiple ligands simultaneously and integrate the signals in such a way to dictate a cellular response. Frequently, transcription factor proteins that regulate specific genes are modified and initiate transcription of new mRNA in response to the original ligand. Such ligand/receptor interactions are well known, and are described in Molecular Biology of the Cell, Alberts et al. 3^{rd} Ed, Garland Publishing, N.Y. 1994.

It is highly desirable to study cellular integration of the signals induced by ligands to further understand critical cell biology questions. Understanding how cells interpret these events will allow the development of new drugs and other means of intervention, such as gene therapy. To understand the components of this complex system, scientists may develop whole cell based assays such that a complete system is tested, or may isolate individual components for individual analysis. A biological assay is a method consisting of two basic steps: 1) contacting a compound with a cell or cellular components; and 2) measuring an effect of the compound on the cell or the cellular components. To achieve specificity one frequently monitors an immediate effect of the ligand-receptor interaction, such as inhibiting extracellular ligand interactions, phosphorylation of a specific peptide, monitoring ionic flux within the cell, or stimulation of new transcription and translation. An example in which the detection mode is tyrosine phosphorylation for assay development is described in “Assay Systems using the CNTF signal transduction pathway” filed Sep. 9, 1994 by Stahl et al. and International Application No. PCT/US94/10163. An example in which the detection means is a nucleic acid is described in “Method of screening for factors that modulate gene expression” filed Oct. 11, 1996 by Cen et al. and International Application No. PCT/US96/16318. An example of whole-cell based assays where the event is monitoring transcription of a exogenous reporter gene is described, for instance, in U.S. Pat. No. 5,436,128 by Harpold et al. and filed Jan. 27, 1993. This example illustrates introduction of an exogenous reporter gene into a cell wherein the reporter gene is encoded downstream of a promoter that becomes activated by signals transduced as a result of extracellular ligand binding to a receptor.

Assays have been developed where chimeric transcription factors responsive to estradiol, activate an exogenous reporter system within a cell as illustrated by Webster N. J. G. et al in “The Hormone-Binding Domain of the Estrogen and Gluococorticoid Receptors Contain an Inducible Transcription Activation Function.” Cell 54: 199-207 (1988). Sourisseau, T. et al. in “Eukaryotic conditional expression system” in Biotechniques 27(1): 106-110 (1999). In this assay the DNA binding domain was GAL4, and utilized the GAL4 promoter upstream of exogenously added chloramphenicol acetyl transferase (CAT). The present invention, for the first known time, illustrates the use of a novel transcription factor construct that binds to a unique DNA sequence of an endogenous gene and activates, through a responsive activating domain on the transcription factor construct, transcription of the endogenous gene in response to signals transduced as a result of extracellular ligand binding to a receptor.

Novel means to measure effects in whole-cell based assays are desirable for scientists who develop biological assays. In particular, assays that are easily adapted for multiple extracellular ligands are expected to reduce the time and expense of drug development. This need is addressed by the present invention that describes a whole-cell based biological assay that utilizes the activation of endogenous genes under control of an exogenous transcription factor.

### SUMMARY OF THE INVENTION

The present invention provides whole-cell biological assays that measure changes in the expression of endogenous genes under control of an exogenously introduced transcription factor. The exogenous transcription factors of the present invention may be designed such that each is activated by specific extracellular ligands. Therefore cells containing one or more exogenous transcription factors of the present invention provide a generic means to which many extracellular ligands may be tested without substantial modification to the assay.

### BRIEF DESCRIPTION OF THE DRAWINGS

FIG. 1—Western blot of chimeric zinc finger constructs in cells detected using an antibody to the affinity tag “FLAG.” “+” indicates cells in the presence of estradiol while “−” indicates cells without estradiol.

FIG. 2—Induction of VEGF protein in HEK 293 cells using an estradiol inducible zinc finger.

FIG. 3—Induction of EPO protein in HEK 293 cells using an estradiol inducible zinc finger.

FIG. 4—Induction of Alkaline phosphatase mRNA using an estradiol inducible zinc finger.

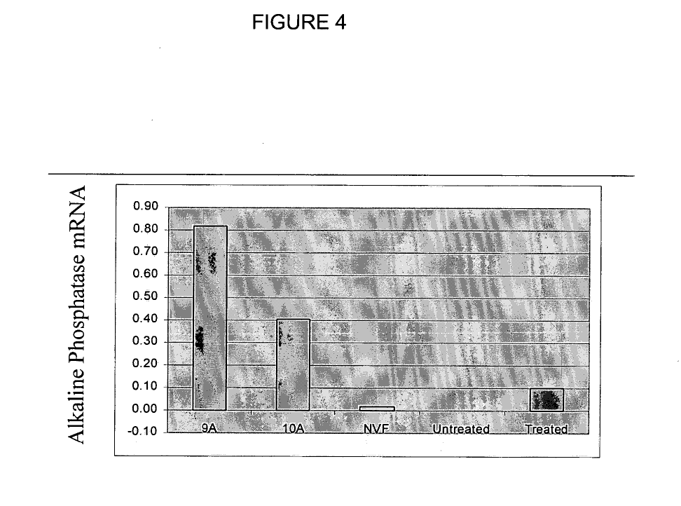

### DETAILED DESCRIPTION

Definitions:

The term “protein domain” as used herein refers to a region of a protein that can fold into a stable three-dimensional structure independent of the rest of the protein. This structure may perform a specific function within the protein including enzymatic activity, creation of a recognition motif for another molecule, or provide necessary structural components for a protein to exist in a particular environment. Protein domains are usually evolutionarily conserved regions of proteins, both within a protein family and within other protein superfamilies that require similar functions.

The term “protein superfamily” as used herein refers to proteins whose evolutionary relationship may be distant by accepted phylogenetic standards, but show similar three-dimensional structure or display unique consensus of critical amino acids.

The term “fusion protein” or “chimera” as used herein refers to a novel protein construct that is the result of combining multiple protein domains or linker regions for the purpose of gaining function of the combined functions of the domains or linker regions. This is most often accomplished by molecular cloning of the nucleotide sequences to result in the creation of a new polynucleotide sequence that codes for the desired protein.

Alternatively, creation of a fusion protein may be accomplished by chemically joining two proteins together. The term “operably linked” means that both domains of the fusion protein maintain their function. For example an inducible activation domain will prevent the transcription factor from being active without proper stimulation. Oppositely a constitutive activation domain would create a transcription factor that would always be fully active.

The term “linker region” or “linker domain” or similar such descriptive terms as used herein refers to stretches of polynucleotide or polypeptide sequence that are used in the construction of a cloning vector or fusion protein. Functions of a linker region can include introduction of cloning sites into the nucleotide sequence, introduction of a flexible component or space-creating region between two protein domains, or creation of an affinity tag for specific molecule interaction. A linker region may be introduced into a fusion protein without a specific purpose, but results from choices made during cloning.

The term “cloning site” or “polycloning site” as used herein refers to a region of the nucleotide sequence contained within a cloning vector or engineered within a fusion protein that has one or more available restriction endonuclease consensus sequences. The use of a correctly chosen restriction endonuclease results in the ability to isolate a desired nucleotide sequence that codes for an in-frame sequence relative to a transcription start site that yields a desirable protein product after transcription and translation. These nucleotide sequences can then be introduced into other cloning vectors, used create novel fusion proteins, or used to introduce specific site-directed mutations. It is well known by those in the art that cloning sites can be engineered at a desired location by silent mutations, conserved mutation, or introduction of a linker region that contains desired restriction enzyme consensus sequences. It is also well known by those in the art that the precise location of a cloning site can be flexible so long as the desired function of the protein or fragment thereof being cloned is maintained.

As used herein, “Expression vectors” are defined herein as DNA sequences that are required for the transcription of cloned copies of genes and the translation of their mRNAs in an appropriate host. Such vectors can be used to express eukaryotic or prokaryotic genes in a variety of hosts including E. coli, blue-green algae, plant cells, insect cells, fungal cells including yeast cells, and animal cells.

As used herein, a “functional derivative” of the DNA binding domain—transactivation domain nucleotide or polypeptide sequence is a nucleotide or polypeptide sequence that possesses a biological activity, either functional or structural, that is substantially similar to the properties described herein. The term “functional derivatives” is intended to include the “fragments,” “variants,” “degenerate variants,” “analogs” and “homologues” of the nucleotide or polypeptide sequence presented. The term “fragment” is meant to refer to any nucleic acid or polypeptide subset of the modules described. The term “variant” is meant to refer to a nucleotide or polypeptide sequence or coding sequence module substantially similar in structure and function to either the entire DNA binding domain—transactivation domain nucleotide or polypeptide sequence molecule or to a fragment thereof. A nucleotide or polypeptide sequence is “substantially similar” to DNA binding domain—transactivation domain nucleotide or polypeptide sequence if both molecules expressed from them have similar structural characteristics or if both molecules possess similar biological properties ie, can be manipulated such that expressed recombinant DNA binding domain—transactivation domain nucleotide or polypeptide sequence. Therefore, if the two molecules possess substantially similar activity, they are considered to be variants even if the structure of one of the molecules is not found in the other or even if the two amino acid sequences are not identical. The term “analog” refers to a molecule substantially similar in function to either the entire nucleotide or polypeptide sequence molecule or to a fragment thereof.

The term “gene” as used herein refers to a contiguous nucleic acid sequence that encodes a discrete heritable characteristic. The term gene encompasses both intronic and exonic sequences within the contiguous sequence.

The term “compound” as used herein refers to an organic or inorganic molecule that has the potential to modulate the specific response of an extracellular ligand. Compounds of the present invention may potentiate or disrupt the response of the extracellular ligand by competitive or noncompetitive means. For example, but not to limit the scope of the current invention, compounds may include small organic or inorganic molecules, synthetic or natural amino acid polypeptides, proteins including monoclonal antibodies, or synthetic or natural nucleic acid sequences.

The term “extracellular ligand” refers to stimuli the cell receives originating from a source other than the cell itself including ligands that bind to cell surface membrane receptors and ligands that diffuse across the cell membrane to an intracellular receptor. This term includes, but is not limited to, polypeptide hormones, growth factors or cytokines, neurotransmitters, mechanical stress, small amino acid and nucleotide (ATP for example) derivatives, or hydrophobic small molecules. This term also encompasses intracellular pores where molecules diffuse from one cell to another in order to transmit information, or where a cell produces an extracellular ligand that then acts in an autocrine fashion. Hormones suitable for use in the present invention may be derived from natural or recombinant sources in the form of crude extract or substantially purified. Purified recombinant hormone is generally preferred. For example, and not by way of limitation, VEGF, TGF, steroid hormones, IFN, IFN, OSM, G-CSF, Leptin, IL-2, IL-7, IL-15, IL-3, IL-5, GM-CSF, EPO, GH, prolactin, TPO, PDGF, CSF-1, and insulin are well known hormones suitable for use in the method of the present invention. Pseudo-hormones may be prepared by matching a soluble polypeptide capable of binding to fusion receptor proteins and inducing conformational changes such that the receptor initiates a signal transduction cascade that terminates in the exogenous transcription factor.

The term “receptor” refers to an adaptor molecule contained on the surface or within a cell that responds to an extracellular ligand and initiates a cellular response that changes the functions of the cell. For example, but not by way of limitation, receptors suitable for use in the present invention include single transmembrane receptors, both enzymatic and nonenzymatic, G-protein coupled seven transmembrane receptors, ion channels responsive to sodium, potassium and calcium, intracellular hormone receptors (which act as transcription factors directly), and integrins. Single transmembrane receptors include those responsive to growth factors and cytokines, and are well known in the art as described by Alberts et al. supra. G-protein coupled seven transmembrane receptors are those that respond to a variety of ligands and are described by Horn, F. et al. “GPCRDB: an information system for G protein-coupled receptors.” Nucleic Acids Res. 26(1): 277-281 (1998). Receptors suitable for use in the present invention may be expressed endogenously on the cell or recombinantly introduced into the cell line. Receptors with no known ligand, known as orphan receptors, may be screened by creating a chimeric protein containing the orphan receptor ligand binding domain with a signal transduction domain of a known receptor, or by monitoring an appropriate signaling pathway determined by the orphan receptor's homology to a known receptor. Also suitable for use in the present invention are fusion receptor proteins comprising an extracellular binding domain and an intracellular domain derived from receptor tyrosine kinases (RTK) or non-RTK provided that the extracellular binding domain is capable of forming oligomers, preferably dimers, after specific interaction with the hormone.

The term “cell” refers to at least one cell, but includes a plurality of cells appropriate for the sensitivity of the detection method. Cells suitable for the present invention may be bacterial, yeast, or eukaryotic. Examples of cell lines that express functional, endogenous human receptors suitable for use in the present invention are shown in Table 1. While human cells that express human receptors are preferred, any cell line derived from any species that expresses a receptor that binds a ligand is suitable for use in the present invention. (eg. Human luteinizing hormone binds to the rat luteinizing hormone receptor with the same affinity as it does to the human luteinizing hormone receptor). In addition to those listed below, it is possible to screen other commercially available cell lines for receptor expression by testing for hormone binding. Further testing for functional receptor expression may be achieved by conducting a differential gene expression assay comparing cells contacted with the hormone with identical cells that have not been contacted by the hormone. Alternatively one could search a commercial cell archive database, such as American Type Culture Collection (ATCC), for known cell lines that express the desired receptor in human and other species.

TABLE 1

| ATCC No. | Cell line | Receptor expressed |
|---|---|---|
| CRL-5822 | NCI-N87 | Muscarinic |
| HTB-140 | Hs 294 T | NGF, interferon |
| CRL-1427 | MG-63 | TGF I, II, TNF-alpha |
| CRL-2062 | DMS 53 | Bombesin, Epidermal Growth |
| | | factor (EGF), TGF |
| CRL-2062 | DMS 53 | Acetylcholine |
| HTB-133 | T-47D | Estradiol, steroid, calcitonin, |
| | | androgen, progesterone |
| HTB-133 | T-47D | Glucocorticoid, prolactin, estrogen |
| CRL-5802 | NCI-H157 | PDGF |
| CRL-1740 | LNCaP.FGC | Androgen, estrogen |
| HB-8065 | Hep G2 | Insulin, insulin-like growth factor II (IGF II) |

DNA encoding an exogenous receptor may be cloned into an expression vector for expression in a recombinant host cell. Recombinant host cells may be prokaryotic or eukaryotic, including but not limited to bacteria such as E. coli, fungal cells such as yeast, plant cells, mammalian cells including but not limited to cell lines of human, bovine, porcine, monkey and rodent origin, and insect cells including but not limited to drosophila and silkworm derived cell lines. Cell lines derived from mammalian species that may be suitable and that are commercially available, include but are not limited to, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), L-cells, and HEK-293 (ATCC CRL1573).

The term “chemical derivative” describes a molecule that contains additional chemical moieties attached to the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

The term “high throughput” as used herein refers to an assay design that allows easy analysis of multiple samples simultaneously, and capacity for robotic manipulation. Another desired feature of high throughput assays is an assay design that is optimized to reduce reagent usage, or minimize the number of manipulations in order to achieve the analysis desired. Examples of assay formats include 96-well or 384-well plates used for liquid handling experiments. It is well known by those in the art that as miniaturization of plastic molds and liquid handling devices are advanced, or as improved assay devices are designed, that greater numbers of samples may be performed using the design of the present invention. Use of 96-well plate assays in the examples is given for illustrative purposes only.

The term “protein transduction” means that protein is introduced into a cell by nonrecombinant means, well known in the art. For example, but not by way of limitation, means of protein transduction include liposome fusion, microinjection, electroporation, microballistic projectile, and by polypeptide mediated transduction (for example Schwarze et al. “In Vivo protein transduction: Delivery of a biologically active protein into the mouse” (1999) Science 285 1569-1572.)

The present invention provides an adaptable whole-cell method to detect the ability of a compound to modulate a signal transduction pathway comprising the steps;
1) contacting a compound and a cell, said cell containing an exogenous transcription factor, and
2) measuring induction of a gene under promotional control of said exogenous transcription factor.

In this method a compound binds to a receptor or any protein within a signaling pathway terminating in the activating domain of the transcription factor and changes the activity of the transcription factor. This results in a change in the activity of the transcription factor. This method is useful to determine compounds that affect any component of a specific signal transduction pathway.

Another embodiment of the present invention contemplates a whole cell method to detect compound modulation of the normal effects of an extracellular ligand towards its receptor comprising the steps:
1) contacting a compound, extracellular ligand, and a cell, said cell being capable of responding to said ligand and containing a exogenous transcription factor, and
2) measuring induction of a gene under promotional control of said exogenous transcription factor.

This method is useful to determine compounds that modulate a receptor by measuring changes of the normal amount of gene product produced by a known amount of a ligand. If the compound is an antagonist, the amount of gene product will decrease, while an agonist compound may increase the level of gene transcription.

In another embodiment of the present invention, the exogenous transcription factor could be designed to upregulate (induce transcription of) a receptor of interest in a cell that normally does not express the receptor. By upregulating the activity of the exogenous transciption factor, the level of receptor can be controlled, thus creating an easy system to generate experimental (stimulated transcription factor) and control cells (unstimulated transcription factor). Thus the method comprises the steps;
1) providing a cell that contains an exogenous transcription factor that upregulates a receptor of interest in a cell; and
2) stimulating the function of the transcription factor with a ligand such that the receptor is produced by the cell.

The amount of time necessary for cellular contact with the extracellular ligand is empirically determined, for example, by running a time course and measuring gene expression from the exogenous transcription factor as a function of time. As a general rule, the activation of the exogenous transcription factor occurs within minutes of cellular receptor engagement and accumulation of endogenous gene products should be detectable within 4-8 hours of cell stimulation, but might not reach maximal until 12-24 hours after ligand contact.

The measurement means of the method of the present invention can be further defined by comparing a cell that has been exposed to an extracellular ligand with one that has not been similarly exposed. Alternatively two cells, one containing the exogenous transcription factor and a second cell identical to the first, but lacking the exogenous transcription factor could be both be contacted with the same extracellular ligand and compound and compared for differences between the two cells. This technique is also useful in establishing the background noise of these assays. One of average skill in the art will appreciate that these control mechanisms also allow easy selection of genes endogenous to the cell and responsive to the exogenous transcription factor.

Transcription factors contain at least two domains, an activation domain and a DNA binding domain. Tan, S. et al, recently reviewed eukaryotic transcription factors in “Eukaryotic transcription factors” Curr Opin Struc Biol 8: 41-48 (1998). The activation domain is the ultimate recipient of the signal pathway propagation, often by phosphorylation or ligand binding. Activated transcription factors then can bind DNA and activate specific genes as described by Zawel, L. et al. “Common themes in assembly and function of eukaryotic transcription complexes” in Ann Rev Biochem 64: 533-561 (1995). If an exogenous transcription factor is introduced into a cell, then activation of the transcription factor will activate genes that are not normally expressed at similar levels in the cell. The present invention contemplates chimeric transcription factors containing an inducible activation domain and a synthetic DNA binding domain. The term “synthetic” means that the DNA binding domain cannot be isolated in nature. For example, the DNA binding domain may comprise polypeptide such as those discovered from peptide libraries that bind to specific DNA sequences, as described by Chen, X. et al. “Selection of peptides that functionally replace a zinc finger in the SPI transcription factor by using a yeast combinatorial library” in Proc. Natl. Acad. Sci. USA 94: 14120-14125 (1997). A preferred synthetic DNA binding domain is one in which a binding domain related to a wild type DNA binding domain has been designed to bind to DNA sequences that are different from those bound by the wild type DNA-binding domain. A highly preferred synthetic DNA binding domain is a zinc finger or a set of linked zinc fingers. Genes encoding designed transcription factors may be recombinantly constructed using methods well known in the art as described in Sambrook et al. (1989), Molecular Cloning: A laboratory manual, 2^{nd} edition (Cold Spring Harbor press, Cold Spring Harbour, New York, N.Y.), and Ausubel et al., Current Protocols in Molecular Biology (1994), (Greene Publishing Associates and John Wiley & Sones, New York, N.Y.). Methods for designing zinc finger transcription factors that bind to specific DNA sequences are described, for example, in U.S. Pat. No. 5,789,538 ZINC FINGER PROTEINS WITH HIGH AFFINITY NEW DNA BINDING SPECIFICITIES, filed Apr. 18, 1997 by Rebar et al., pending WIPO publication PCT/US98/10801 ZINC FINGER PROTEIN DERIVATIVES AND METHODS THEREFOR, filed May 27, 1998 by Barbas et al., pending WIPO publication PCT/GB98/01510 NUCLEIC ACID BINDING POLYPEPTIDE LIBRARY, filed May, 23, 1997 by Choo et al., and WIPO publication WO 00/42219 SELECTION OF SITES FOR TARGETING BY ZINC FINGER PROTEINS AND METHODS OF DESIGNING ZINC FINGER PROTEINS TO BIND TO PRESELECTED SITES, Jul. 20, 2000.

The selection of a DNA sequence to which a modified transcription factor is designed is (A) within about 2,000 base pairs upstream of the native transcription unit start site; or (B) any intronic or exonic sequence within the gene. The sequence is expressed in at least one copy as illustrated by Schatt, M. D. et al. “A single DNA-binding transcription factor is sufficient for activation from a distant enhancer and/or from a promoter position” EMBO J. 9: 481-487 (1990). Preferred DNA sequences are within about 500 base pairs upstream of the transcription unit. Most preferred DNA sequences are those that contain a unique 9-bp sequence corresponding to a synthetic zinc finger binding domain within 2,000 bp of the transcription unit. Since a 9-bp sequence will occur infrequently within the promoter region of genes within the human genome, this selection allows a higher selectivity in the activation of the endogenous genes. DNA sequences greater than 9 bp can be used, as well.

Endogenous genes that are suitable for use in the present invention are those that are not normally expressed at a high level in the cell so that even a modest increase of expression can be measured as a reasonable signal over background level. Many tissue-specific genes fulfill this criterion. Tissue-specific genes can be determined, for example, by differential gene expression. Another consideration in choosing transcription factor target genes in the present invention is the ease of measurement. Secreted proteins could be measured by colorimetric or fluorometric ELISAs. Enzymes with chromogenic or fluorogenic substrates are excellent assay targets. Preferred genes are secreted enzymes such as secretory placental alkaline phosphatase (SPAP) or secreted proteases, and cell surface proteins such as membrane-anchored alkaline phosphatase or “CD antigens” as defined by the International Workshops on Human Leukocyte Differentiation Antigens. Choice of an appropriate endogenous gene is made based on the cell line under analysis. Alkaline phosphatase is a preferred endogenous gene because it is rarely expressed in the cell outside placental cells, and is readily detected using commercially available detection systems. Other preferred enzymes that may be expressed include myeloperoxidase or serine proteases. Where a secreted enzyme, such as alkaline phosphatase is not desired, a different endogenous gene may be selected. For example, if a cell other than CD8+ T cell were under study, a suitable gene could be CD8, which could be analyzed by flow cytometric analysis or fluorescent cell imaging analysis of its expression at the cell surface.

To use the endogenous genes to report cell activation, transcription activation domains that can be modulated are used in chimeric transcription factors. Choice of transcription activation domains depends on the signaling pathway to be assayed. For example, to report growth factors activation (for example, but not limited to, PDGF, VEGF), one could introduce the ternary complex factor Elk-1 C-terminal region—ZFP fusion protein into growth factor responsive cells. The Elk-1 C-terminal region (a.a. 307-428) has multiple phosphorylation sites for MAP kinases and it functions as a regulated transcriptional activation domain whose activity in the transfected cells is dependent on growth factor stimulation. The amount of endogenous gene expression, which is measured in a high-throughput manner, is therefore proportional to the extent of pathway activation. Similarly regulated transcription domains are available for cytokine pathways (STAT C-terminal region) and pathways that generate second messengers such as Ca++ and cAMP (CREB or ATF2 kinase inducible domain). G-protein coupled receptors (GPCR) often activate adenyl cyclase that results in generation of the second messenger cAMP. cAMP then stimulates the activation domain of CREB.

In addition, chimeric proteins can be made to report pathways in which transcription events are modulated by ligand binding. For example, steroid receptors have a hormone-binding domain that interacts with hsp90. Upon ligand binding, hsp90 dissociates and the DNA binding domain, which is also of Zn-finger type, is exposed and direct target gene activation. Cells expressing a chimeric intracellular receptor whose DNA binding region is replaced by a novel DNA binding domain will respond to hormone ligands by activating the endogenous gene the engineered DNA binding domain recognizes.

It is also possible to use endogenous gene induction to report cell activation events based on protease activation. A general strategy would be to fuse AP-ZFP-VP 16 (a strong transcription activator) to a dominant membrane localization sequence (for example, the first 10 a.a. of Lck) with a linker peptide recognized by an inducible protease. The chimeric transcription factor resides in cytoplasmic membrane and can not activate target genes until it is processed by the induced protease and released from the cell membrane. One example is to use calpain cleavage to report intracellular increase of Ca⁺⁺ due to GPCR or ion channel activation: chimeric protein with N-terminus-Lck dual acylation sequence, calpain substrate site, AP-ZFP and VP16-C-terminus. Calpain's activity is strictly Ca++dependent and its cleavage on the linker peptide will release the AP-ZFP-VP16 to the nucleus and activate endogenous alkaline phosphatase expression.

In cases where the nuclear translocation signal is better characterized than transcription activation signal during cell induction, it is possible to fuse the nuclear translocation domain of the signaling protein to the AP-ZFP-VP16. Cell activation will now lead to nuclear accumulation of the chimeric transcription factor and increased level of alkaline phosphatase activity. An example is NF-kB.

The measurement means suitable for the method of the present invention comprises measuring changes in the induction level of a naturally occurring gene product in a cell. Preferred measurement means include changes in the quantity of mRNA, intracellular protein, cell surface protein, or secreted protein. Levels of mRNA are detected by reverse transcription polymerase chain reaction (RT-PCR) or by differential gene expression. Immunoaffinity, ligand affinity, or enzymatic measurement quantitates levels of protein in host cells. Protein-specific affinity beads or specific antibodies are used to isolate for example ³⁵S-methionine labelled or unlabelled protein. Labelled protein is analyzed by SDS-PAGE. Unlabelled protein is detected by Western blotting, cell surface detection by fluorescent cell sorting, cell image analysis, ELISA or RIA employing specific antibodies. Where the protein is an enzyme, the induction of protein is monitored by modification of a fluorogenic or colorimetric substrate.

Preferred detection means for cell surface protein include flow cytometry or fluorescent cell imaging. In both techniques the protein of interest is localized at the cell surface, labeled with a specific fluorescent probe, and detected via the degree of cellular fluorescence. In flow cytometry, the cells are analyzed in a suspension, whereas in cellular imaging techniques, a field of cells is compared for relative fluorescence. For methods where an intracellular protein is measured, for example by total amount or spacial translocation, cells may be fixed and analyzed by these techniques.

A preferred detection means for secreted proteins that are enzymes such as alkaline phosphatase or proteases, would be fluorescent or colorimetric enzymatic assays. Fluorescent/luminescent/color substrates for alkaline phosphatase are commercially available and such assays are easily adaptable to high throughput multiwell plate screen format. Fluorescent energy transfer based assays are used for protease assays. Fluorophore and quencher molecules are incorporated into the two ends of the peptide substrate of the protease. Upon cleavage of the specific substrate, separation of the fluorophore and quencher allows the fluorescence to be detectable. When the secreted protein can be measured by radioactive methods, scintillation proximity technology can be used. The substrate of the protein of interest is immobilized either by coating or incorporation on a solid support that contains a scintillant material. A radioactive molecule, brought in close proximity to the solid phase by enzyme reaction, causes this material to become excited and emit visible light. Emission of light forms the basis of detection of successful ligand/target interaction, and is measured by an appropriate monitoring device. An example of a scintillation proximity assay is disclosed in U.S. Pat. No. 4,568,649, issued Feb. 4, 1986. Materials for these types of assays are commercially available and are well known in the art.

A preferred detection means where the endogenous gene results in phenotypic cellular structural changes is statistical image analysis of the cellular morphology or intracellular phenotypic changes. For example, but not by way of limitation, a cell may change morphology such as rounding versus remaining flat against a surface, or may become growth-surface independent and thus resemble transformed cell phenotype well known in the art of tumor cell biology, or a cell may produce new outgrowths. Phenotypic changes that may occur intracellularly include cytoskeletal changes, alteration in the endoplasmic reticulum/Golgi complex in response to new gene transcription, or production of new vesicles.

Where the endogenous gene encodes a soluble intracellular protein, changes in the endogenous gene may be measured by changes of the specific protein contained within the cell lysate. The soluble protein may be measured by the methods described herein.

Where enhanced expression of an endogenous gene is desired, for instance in cells that repress the endogenous gene, the cell may be modified to modulate the repressed state. Exposing the cell with compounds that cause demethylation or histone deacetylation increases expression of genes that are normally silenced (Cameron, E. E. et al (1999) Nat Genet 21(1) 103-107). Examples of two known drugs useful in the present methods are trichostatin A (TSA), which in an inhibitor of histone deacetylase, and 5-aza-2′deoxycytidine (5Aza-dC), which demethylates DNA. Alternatively a cell could be caused to express higher than normal levels of a demethylation gene, for example as described by Bhattacharya, SK et al (1999) Nature 397(6720) 579-583. Higher expression of a demethylation gene could be accomplished by methods well known in the art including recombinant introduction or by natural or directed selection methods. In additional embodiments, inhibitors of phosphatases, ubiquitin hydrolases and ADP ribosylases can be used in similar fashion.

The present invention also has the objective of providing suitable topical, oral, systemic, and parenteral pharmaceutical formulations for use in novel methods of treatment of the present invention. The compositions containing compounds or modulators identified according to this invention as the active ingredient for use in the modulation of receptors can be administered in a wide variety of therapeutic dosage forms in conventional vehicles for administration. For example, the compounds or modulators can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a receptor ligand-modulating agent.

The daily dosage of the products may be varied over a wide range from 0.01 to 1,000 mg per patient, per day. For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. The dosages of the receptor modulators are adjusted when combined to achieve desired effects. On the other hand, dosages of these various agents may be independently optimized and combined to achieve a synergistic result wherein the pathology is reduced more than it would be if either agent were used alone.

Advantageously, compounds or modulators of the present invention may be administered in a single daily dose, or the total daily dosage may be administered in divided doses of two, three or four times daily. Furthermore, compounds or modulators for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds or modulators of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

In the methods of the present invention, the compounds or modulators herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or -lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents that may be employed include glycerin and the like. For parenteral administration, sterile suspensions and solutions are desired. Isotonic preparations, which generally contain suitable preservatives, are employed when intravenous administration is desired.

Topical preparations containing the active drug component can be admixed with a variety of carrier materials well known in the art, such as, eg., alcohols, aloe vera gel, allantoin, glycerine, vitamin A and E olis, mineral oil, PPG2 myristyl propionate, and the like, to form, eg., alcoholic solutions, topical cleansers, cleansing creams, skin gels, skin lotions, and shampoos in cream or gel formulations.

The compounds identified by the method of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds identified by the method of the present invention may also be delivered by the use of monoclonal antibodies as individual carriers to which the compound molecules are coupled. The compounds or modulators of the present invention may also be coupled with soluble polymers as targetable drug carriers. Such polymers can include polyvinyl-pyrrolidone, pyran copolymer, polyhydroxypropylmethacryl-amidephenol, polyhydroxy-ethylaspartamidephenol, or polyethyl-eneoxidepolylysine substituted with palmitoyl residues. Furthermore, the compounds or modulators of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For oral administration, the compounds identified by the methods of the present invention may be administered in capsule, tablet, or bolus form or alternatively they can be mixed in the animals feed. The capsules, tablets, and boluses are comprised of the active ingredient in combination with an appropriate carrier vehicle such as starch, talc, magnesium stearate, or di-calcium phosphate. These unit dosage forms are prepared by intimately mixing the active ingredient with suitable finely-powdered inert ingredients including diluents, fillers, disintegrating agents, and/or binders such that a uniform mixture is obtained. An inert ingredient is one that will not react with the compounds or modulators and which is non-toxic to the animal being treated. Suitable inert ingredients include starch, lactose, talc, magnesium stearate, vegetable gums and oils, and the like. These formulations may contain a widely variable amount of the active and inactive ingredients depending on numerous factors such as the size and type of the animal species to be treated and the type and severity of the infection. The active ingredient may also be administered as an additive to the feed by simply mixing the compound with the feedstuff or by applying the compound to the surface of the feed. Alternatively the active ingredient may be mixed with an inert carrier and the resulting composition may then either be mixed with the feed or fed directly to the animal. Suitable inert carriers include corn meal, citrus meal, fermentation residues, soya grits, dried grains and the like. The active ingredients are intimately mixed with these inert carriers by grinding, stirring, milling, or tumbling such that the final composition contains from 0.001 to 5% by weight of the active ingredient.

The compounds identified in the methods of the present invention may alternatively be administered parenterally via injection of a formulation consisting of the active ingredient dissolved in an inert liquid carrier. Injection may be either intramuscular, intraruminal, intratracheal, or subcutaneous. The injectable formulation consists of the active ingredient mixed with an appropriate inert liquid carrier. Acceptable liquid carriers include the vegetable oils such as peanut oil, cotton seed oil, sesame oil and the like as well as organic solvents such as solketal, glycerol formal and the like. As an alternative, aqueous parenteral formulations may also be used. The vegetable oils are the preferred liquid carriers. The formulations are prepared by dissolving or suspending the active ingredient in the liquid carrier such that the final formulation contains from 0.005 to 10% by weight of the active ingredient.

Topical application of the compounds identified by the methods of the present invention is possible through the use of a liquid drench or a shampoo containing the instant compounds or modulators as an aqueous solution or suspension. These formulations generally contain a suspending agent such as bentonite and normally will also contain an antifoaming agent. Formulations containing from 0.005 to 10% by weight of the active ingredient are acceptable. Preferred formulations are those containing from 0.01 to 5% by weight of the instant compounds or modulators.

Abbreviations:
ACTH Adrenocorticotropic hormone
Jak Janus Kinase
STAT signal transducer and activator of transcription
IL interleukin
G-CSF granulocyte-colony stimulating factor
GM-CSF granulocyte-macrophage colony stimulating factor
IFN interferon
EGF epidermal growth factor
PDGF platelet derived growth factor
EPO erythropoietin
TPO thrombopoietin
GH growth hormone
LH Luteinizing hormone
PTK protein tyrosine kinase
SH2 src homology 2 domain
SH3 src homology 3 domain
VEGF Vascular endothelial growth factor
TGF Transforming growth factor D family hormones
Hsp Heat Shock Protein

The following examples illustrate the present invention without, however, limiting the same thereto.

### EXAMPLE 1

Jak-STAT Receptor Pathway with Chimeric Transcription Factor—AP Detection

HeLa, a cell line that endogenously expresses IFN receptor and is responsive to IFN is transiently or stably transfected with a chimeric transcription factor. The chimeric transcription factor is composed of a DNA binding domain that binds upstream of the human alkaline phosphatase gene inducing expression of the alkaline phosphatase gene, and a STAT1 transcription activating domain (aa 713-750) responsive to IFN.

A small aliquot of a compound and 1 nanomolar IFN are added to a microwell plate containing approximately 5×105 cells containing the chimeric transcription factor (Cell/+TF) and simultaneously against the same amount of cells without the transcription factor (Cell/−TF), and incubated at 37° C. for 8 hours, appropriate controls are without compound and/or without IFN. Then supernatant from each well is assayed for modulation of the IFN signaling pathway by the measurement of an increase in alkaline phosphatase activity based upon the hydrolysis of the substrate phosphate ester of p-nitrophenol (PNPP) to p-nitrophenol (PNP) and phosphate by measuring the increase in absorbance at 405 nm.

Cell/+TF not exposed to a modulating compound respond to IFN and generate an increased absorbance at 405 nm. Cell/−TF not exposed to a modulating compound respond to INF but do not generate an increased absorbance at 405 nm. Cell/+TF exposed to modulating compounds are compared to these cells to determine if the compound affected the INF signaling pathway.

G Protein-Coupled Receptor with Chimeric Transcription Factor—Serine Protease Detection

Human 293 cells stably expressing dopamine receptor D1 are selected by antibiotic resistance gene co-expression. These cells are further transfected with a chimeric transcription factor. The chimeric transcription factor is composed of a DNA binding domain that binds upstream of human prostate specific antigen (PSA) protease and a CREB activating domain responsive to dopamine via the cAMP pathway.

A small aliquot of a compound and 10 micromolar dopamine are added to a microwell plate containing 5×10⁵ cells containing the chimeric transcription factor (Cell/+TF) and simultaneously against the same amount of cells (Cell/−TF), and incubated at 37° C. for 4 hours. The supernatant from each well is assayed for modulation of the serotonin receptor signaling pathway by measurement of aminolytic activities of the PSA protease. Aminolytic activity is monitored by a FRET-based method. A PSA specific substrate peptide is synthesized with EDENS (quencher) on the N-terminal amino acid and fluorescein on the C-terminal amino acid, cleavage by PSA will release the EDENS and lead to increase of fluorescence in the media. Cell/+TF not exposed to a modulating compound respond to dopamine and generate an increased fluorescence at 523 nm. Cell/−TF not exposed to a modulating compound respond to dopamine but do not generate an increased fluorescence at 525 nm. Cell/+TF exposed to modulating compounds are compared to these cells to determine if the compound affected the dopamine signaling pathway.

Receptor Tyrosine Kinase Pathway with Chimeric Transcription Factor—Cell Surface CD8 Detection

3T3, a cell line that endogenously expresses PDGF receptor and is responsive to PDGF is transiently transfected with a chimeric transcription factor. The chimeric transcription factor is composed of a DNA binding domain that binds upstream of human T-cell surface glycoprotein CD8 and an Elk-1 activating domain, which is responsive to PDGF.

A small aliquot of a compound and 1 nanomolar PDGF are added to a microwell plate containing 5×10⁵ cells containing the chimeric transcription factor (Cell/+TF) and simultaneously against the same amount of cells (Cell/−TF), and incubated at 37° C. for 4 hours. Then cells from each well are assayed for modulation of the PDGF (whose receptor is a protein tyrosine kinase) signaling pathway by the measurement of cell surface expression of CD8.

Approximately 50 ug of antibody specific to CD8 and coupled with a florescent probe (Beckman Coulter, Fullerton Calif.) is added to each well and incubated for 30 minutes at 37° C. Then the cells are analyzed using a COULTER® EPICS® flow cytometer to determine the degree of bound florescent antibody.

Cell/+TF not exposed to a modulating compound respond to PDGF and generate an increased fluorescence. Cell/−TF not exposed to a modulating compound respond to PDGF but do not generate an increased fluorescence. Cell/+TF exposed to modulating compounds are compared to these cells to determine if the compound affected the PDGF signaling pathway.

Chimeric Intracellular Receptor—Placental Alkaline Phosphatase

A chimeric intracellular receptor is recombinantly constructed using the ligand binding domain of the estrogen receptor and a DNA binding domain specific for the promoter region of placental membrane-associated alkaline phosphatase. The original transactivation domain of estrogen receptor N-terminus of the DNA binding domain is not perturbed. This cDNA is recombinantly cloned in-frame and in the correct orientation into a pCIneo vector.

Co-transfection of any vector containing chimeric intracellular receptor with a drug selection plasmid including, but not limited to G-418, aminoglycoside phosphotransferase; hygromycin, hygromycin-B phosphotransferase; APRT, xanthine-guanine phosphoribosyl-transferase, allow for the selection of stably transfected clones. Levels of chimeric intracellular receptor are quantitated by the assays described herein.

Recombinant plasmids containing the chimeric intracellular receptor construct and a drug selection gene are used to transfect mammalian COS or CHO cells by CaPO4-DNA precipitation. Stable cell clones are selected by growth in the presence of G-418. Single G418 resistant clones are isolated and shown to contain the intact chimeric intracellular receptor gene. Clones containing the cDNAs are analyzed for expression of the endogenous gene upon stimulation with estradiol.

A small aliquot of a compound and 100 nanomolar estradiol are added to a microwell plate containing stably transfected COS cells containing the chimeric transcription factor (Cell/+TF) and simultaneously against COS cells (Cell/−TF), and incubated at 37° C. for 60 minutes. Then cells from each well are assayed for modulation of the estrogen receptor signaling by the measurement of cell surface expression of alkaline phosphatase.

Approximately 50 ug of antibody specific to alkaline phosphatase (Amersham) and coupled with a florescent probe (Beckman Coulter, Fullerton Calif.) is added to each well and incubated for 30 minutes at 37° C. Then the cells are analyzed using a COULTER® EPICS® flow cytometer to determine the degree of bound florescent antibody.

COS/+TF not exposed to a modulating compound respond to estradiol and generate an increased fluorescence. COS/−TF not exposed to a modulating compound do respond to estradiol and do not generate an increased fluorescence. Cel/+TF exposed to modulating compounds are compared to these two populations of cells to determine if the compound affected the estrogen receptor signaling.

### EXAMPLE 2

Chimeric Intracellular Receptor Induction of Endogenous VEGF

Table 2 provides the name and description of chimeric transcription factors used to test the ability to regulate the DNA binding domain designed to activate two different proteins. These use of these constructs is further described in Examples 2 and 3.

TABLE 2

| Construct | Description |
|---|---|
| pCV-VF1 | VP16 transactivating domain - strong constitutive activator |
| | VEGF promoter binding domain |
| pNER-VF1 | Estradiol transactivating domain - ligand regulated activator |
| | VEGF promoter binding domain |
| pNERF-VF1 | Estradiol transactivating domain with a FLAG epitope |
| | VEGF promoter binding domain |
| pERF-EP2C | Estradiol transactivating domain with a FLAG epitop |
| | EPO promoter binding domain |
| pCV-EP2C | VP16 transactivating domain |
| | EPO promoter binding domain |

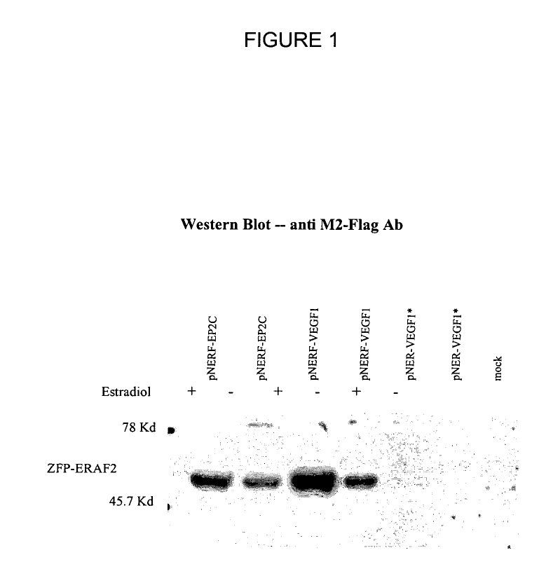

A chimeric transcription factor was recombinantly constructed that contained an intracellular receptor activating domain (transcription activation domain of the human estrogen receptor 1 alpha (Leu308-Val 595) and a DNA binding domain specific for the promoter region of VEGF. This cDNA was recombinantly cloned in-frame and in the correct orientation into a mammalian expression vector. Then HEK 293 cells were transiently transfected in a 6-well format. Each well was transfected using 1 ug DNA+5 ug Lipofectamine. Transfection was performed over 4 hours at 37° C./5% CO₂. Cells were then fed with a 1:1 ratio of 20% FBS in DMEM to yield a final concentration of 10% FBS in DMEM. The next morning, supernatant was removed and cells were re-fed with fresh 10% FBS media, with or without 1 uM Estradiol (Sigma). 24 hours later, supernatants were harvested and assayed for secreted VEGF protein using commercially available Human VEGF ELISA kits (R&D Systems). The relative expression levels of the ZFP-ERLBD protein was determined by Western blot assay, as shown in FIG. 1.

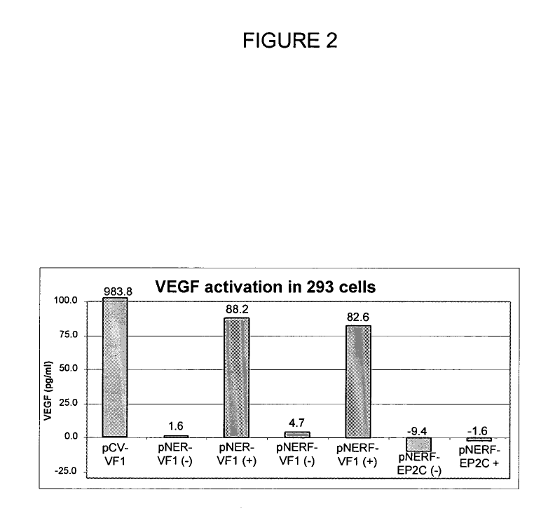

VEGF was not expressed endogenously or activated by estradiol in HEK293 cells as demonstrated by the lack of VEGF produced by cells expressing a chimeric zinc finger directed for the EPO promoter, as seen in FIG. 2. The chimeric zinc finger proteins directed towards the VEGF promoter demonstrated an estradiol-induced increase in VEGF protein by 20-55 fold as seen by comparing either pNER-VF1+/− estradiol or pNERF-VF1+/−estradiol. Additionally the presence of the Flag epitope did not appreciably affect the ability of the transcription factor to induce expression of VEGF. The level of VEGF protein production by the zinc finger is due to the composition of the activation domain, as demonstrated by the higher level of VEGF produced by a chimeric zinc finger containing the strong constitutive VP-16 activation domain, compared to that of the estradiol bound—estradiol transactivating domain. Regardless, constructs containing a nuclear receptor activation domain (estrogen) demonstrate an extraordinarily wide dynamic range in which one can assign different levels of cell activation under influences of activators and inhibitors.

### EXAMPLE 3

Chimeric Intracellular Receptor Induction of Endogenous EPO

A chimeric intracellular receptor was recombinantly constructed using the ligand binding domain and transcription activation domain of the human estrogen receptor 1 alpha (Leu308-Val 595) and a DNA binding domain specific for the promoter region of EPO. This cDNA was recombinantly cloned in-frame and in the correct orientation into a mammalian expression vector. A test of this methodology in 293 cells by transient transfection was done in a 6-well format. Each well was transfected using 1 ug DNA+5 ug Lipofectamine. Transfection was performed over 4 hours at 37° C./5% CO₂. Cells were then fed with a 1:1 ratio of 20% FBS in DMEM to yield a final concentration of 10% FBS in DMSO. The next morning, supernatant was removed and cells were re-fed with fresh 10% FBS media, with or without 1 uM Estradiol (Sigma). 24 hours later, supernatants were harvested and assayed for secreted EPO protein using commercially available Human EPO ELISA kits (R&D Systems). The relative expression levels of the ZFP-ERLBD proteins was determined by Western blot assay.

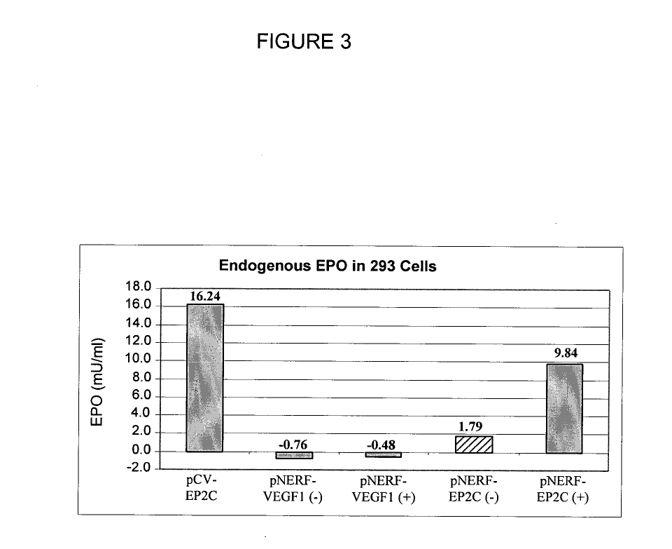

EPO was not expressed endogenously or activated by estradiol in HEK293 cells as demonstrated by the lack of EPO produced by cells expressing a chimeric zinc finger directed for the VEGF promoter, as seen in FIG. 3. The chimeric zinc finger proteins directed towards the EPO promoter demonstrated estradiol-induced increase in EPO protein by approximately 5 fold as seen by comparing pNERF-E2C+/−estradiol. The level of EPO protein production by the zinc finger is due to the composition of the activation domain, as demonstrated by the higher level of EPO produced by a chimeric zinc finger containing the strong constitutive promoter VP-16 compared to that of the estradiol bound—estradiol transactivating domain. However in this case the relative amount of EPO produced is similar comparing the constitutive activation domain that yielded production of 16.24 mU/mL versus 9.84 mU/mL for the regulated activation domain.

### EXAMPLE 4

Chimeric Intracellular Receptor Induction of Alkaline Phosphatase

A chimeric zinc finger was prepared using an estradiol inducible domain and a DNA binding domain specific for the endogenous alkaline phosphatase gene.

As Illustrated in

1. A nucleic acid that encodes a chimeric transcription factor comprising:
(a) an inducible active tion domain responsive to signal transduction from an extracellular ligand; and
(b) a synthetic DNA binding domain that binds a nucleic acid sequence and activates transcription of an endogenous gene
wherein the activation domain is operably linked to the DNA binding domain such that activity of the transcription factor can be regulated.

2. The nucleic acid of claim 1 wherein the activation domain is responsive to a signal transduction pathway selected from the group consisting of Jak-STAT, MAP kinases, phosphatidyl inositol/Ca++, and cyclic nucleotides.

3. The nucleic acid of claim 1 wherein the activation domain is the ligand binding domain of an intracellular receptor.

4. The nucleic acid of claim 1 wherein the synthetic DNA binding domain contains at least one non-zinc finger polypeptide.

5. The nucleic acid of claim 1 wherein the synthetic DNA binding domain contains at least one modified zinc finger.

6. The nucleic acid of claim 5 wherein the synthetic DNA binding domain binds to a DNA sequence within about 2,000 base pairs of the transcription unit.

7. The nucleic acid of claim 5 wherein the synthetic DNA binding domain binds to a unique 9 base pair sequence within about 2,000 base pairs of the transcription unit.

8. The nucleic acid of claim 5 wherein the synthetic DNA binding domain binds to a sequence within the gene.

9. The nucleic acid of claim 1 wherein the activation domain is responsive to a Cytokine receptor signal transduction pathway and the synthetic DNA binding domain contains at least 1 modified zinc finger.

10. The nucleic acid of claim 1 wherein the activation domain is responsive to a Growth Factor signal transduction pathway and the synthetic DNA binding domain contains at least 1 modified zinc finger.

11. The nucleic acid of claim 1 wherein the activation domain is responsive to a G-protein coupled receptor signal transduction pathway and the synthetic DNA binding domain contains at least 1 modified zinc finger.

12. The nucleic acid of claim 1 wherein the activation domain is the ligand binding domain of an intracellular receptor and the synthetic DNA binding domain contains at least 1 modified zinc finger.

13. An expression vector comprising a nucleic acid selected from the group consisting of:
(a) claim 1
(b) claim 2
(c) claim 3
(d) claim 4
(e) claim 5
(f) claim 6
(g) claim 7
(h) claim 8
(i) claim 9
(j) claim 10
(k) claim 11; and
(l) claim 12.

14. A cell containing an expression vector of claim 13.

15. The cell of claim 14 wherein the cell is of mammalian origin.

16. The cell of claim 15 wherein the cell is of human origin.

17. The cell of claim 14 wherein the cell is a bacterial cell.

18. The cell of claim 14 wherein the cell is a yeast cell.

19. The cell of claim 14 wherein the cell is an insect cell.

20. The cell of claim 14 wherein the cell is a plant cell.

21. A process for expression of a chimeric transcription factor in a recombinant host cell, comprising:
(a) transferring the expression vector of claim 13 into suitable host cells; and
(b) culturing the host cells under conditions that allow expression of the chimeric transcription factor from the expression vector.

22. The process of claim 21 further comprising
(c) isolating the transcription factor from the cell.

23. A polypeptide comprising a chimeric transcription factor isolated by the process of claim 22.

24. A monospecific antibody that immunologically reacts to the chimeric transcription factor of claim 23.

25. A cell comprising the polypeptide of claim 23

26. The cell of claim 25 wherein the polypeptide is introduced by liposome, electroporation, microinjection, micro-ballistic projectile, or polypeptide mediated transduction.

27. A method comprising the steps:
a) contacting a compound with a cell of claim 14 wherein the cell expresses a chimeric transcription factor;
b) measuring expression of a gene under promotional control of said transcription factor;
c) determining the effect of the compound on the amount of gene expression.

28. The method of claim 27 wherein the cell is modified to modulate the repressed state of the endogenous gene.

29. The method of claim 28 wherein the cell is modified by pre-exposure to drugs selected from a group consisting of: trichostatin A (TSA), and 5-aza-2′deoxycytidine (5-Aza-dC).

30. The method of claim 27 wherein step (b) measures the level of mRNA of the endogenous gene.

31. The method of claim 27 wherein step (b) measures the level of protein produced by the endogenous gene.

32. The method of claim 27 wherein the endogenous gene is an enzyme and step (b) is measured by monitoring the amount of enzyme activity of the endogenous gene.

33. The method of claim 33 wherein the endogenous gene is an enzyme selected from the group consisting of alkaline phosphatase, myeloperoxidase, and a serine protease.

34. The method of claim 27 wherein the induced endogenous gene produces a cell surface protein.

35. The method of claim 34 wherein the cells surface protein is selected from the group consisting of a CD antigen, a non-CD antigen transmembrane protein, and placental alkaline phosphatase.

36. The method of claim 27 wherein the cells are lysed and the product of the induction of the endogenous gene is measured in the cell lysate.

37. The method of claim 27 wherein the induction of the endogenous gene results in a change in cellular phenotype that is measured in step (b).

38. The method of claim 37 wherein the cellular phenotype is translocation of a protein from one cellular component to a different cellular component.

39. A method comprising the steps:
a) contacting a compound, an extracellular ligand and a cell of claim 14, said cell being capable of responding to said ligand and containing a chimeric transcription factor;
b) measuring expression of an endogenous gene under transcription control of said exogenous transcription factor; and
c) determining the effect of the compound on the amount of gene expression.

40. The method of claim 39 wherein the cell is modified to modulate the repressed state of the endogenous gene.

41. The method of claim 40 wherein the cell is modified by pre-exposure to drugs selected from a group consisting of: trichostatin A (TSA), and 5-aza-2′deoxycytidine (5-Aza-dC).

42. The method of claim 39 wherein step (b) measures the level of mRNA of the endogenous gene.

43. The method of claim 39 wherein step (b) measures the level of protein produced by the endogenous gene.

44. The method of claim 39 wherein the endogenous gene is an enzyme and step (b) is measured by monitoring the amount of enzyme activity of the endogenous gene.

45. The method of claim 44 wherein the endogenous gene is an enzyme selected from the group consisting of alkaline phosphatase, myeloperoxidase, and a serine protease.

46. The method of claim 39 wherein the induced endogenous gene produces a cell surface protein.

47. The method of claim 46 wherein the cells surface protein is selected from the group consisting of a CD antigen, a non-CD antigen transmembrane protein, and placental alkaline phosphatase.

48. The method of claim 39 wherein the cells are lysed and the product of the induction of the endogenous gene is measured in the cell lysate.

49. The method of claim 39 wherein the induction of the endogenous gene results in a change in cellular phenotype that is measured in step (b).

50. The method of claim 49 wherein the cellular phenotype is translocation of a protein from one cellular component to a different cellular component.

51. A nucleic acid that encodes a chimeric transcription factor comprising:
(a) an inducible activation domain responsive to signal transduction from an extracellular ligand; and
(b) a synthetic DNA binding domain that binds to a nucleic acid sequence and activates transcription of an endogenous receptor gene
wherein the activation domain is operably linked to the DNA binding domain such that activity of the transcription factor can be regulated.

52. An expression vector comprising a nucleic acid of claim 51.

53. A cell containing the expression vector of claim 52.

54. The cell of claim 53 wherein the cell is of mammalian origin.

55. The cell of claim 54 wherein the cell is of human origin.

56. The cell of claim 53 wherein the cell is a bacterial cell.

57. The cell of claim 53 wherein the cell is a yeast cell.

58. The cell of claim 53 wherein the cell is an insect cell.

59. The cell of claim 53 wherein the cell is a plant cell.

60. A process for expression of a chimeric transcription factor in a recombinant host cell, comprising:
(a) transferring the expression vector of claim 52 into suitable host cells; and
(b) culturing the host cells under conditions that allow expression of the chimeric transcription factor from the expression vector.

61. The process of claim 60 further comprising
(c) isolating the transcription factor from the cell.

62. A polypeptide comprising a chimeric transcription factor isolated by the process of claim 61.

63. A cell wherein the polypeptide of claim 62 is introduced by nonrecombinant means.

64. The cell of claim 63 wherein the polypeptide is introduced by liposome, electroporation, microinjection, micro-ballistic projectile, or polypeptide mediated transduction

65. A monospecific antibody that immunologically reacts to the chimeric transcription factor of claim 62.

66. A method to modulate the amount of a receptor comprising the steps:
a) providing a cell of claim 53 wherein the cell contains an inducible transcription factor that up-regulates a receptor of interest in the cell; and
b) stimulating the function of the transcription factor with an extracellular ligand such that the receptor is produced by the cell
wherein the amount the receptor is modulated by the activity of the transcription factor.

67. A method comprising the steps:
a) contacting a compound with an un-stimulated cell of claim 53 and contacting a compound with a stimulated cell of claim 53 such that the stimulated cell expresses a receptor;
b) measuring the activity of the receptor in the un-stimulated cell and the stimulated cell; and
c) determining the effect of the compound on the activity of the receptor.

68. A method comprising the steps:
a) contacting a compound and an extracellular ligand with an unstimulated cell of claim 53 and contacting a compound and an extracellular ligand with a stimulated cell of claim 53 such that the stimulated cell expresses a receptor;
b) measuring the activity of the receptor in the unstimulated cell and the stimulated cell; and
c) determining the effect of the compound on the activity of the receptor.

69. A nucleic acid that encodes a chimeric transcription factor comprising:
a) a constitutively active domain;
b) a synthetic DNA binding domain that binds to a nucleic acid sequence and activates transcription of an endogenous gene; and
c) a membrane anchoring domain that contains a protease cleavage site
wherein the constitutively active domain is operably linked to the DNA binding domain such that the transcription factor is active in an unregulated fashion.

70. An expression vector containing the nucleic acid of claim 69.

71. A cell that contains the expression vector of claim 70.

72. A method comprising the steps:
a) contacting a compound with a cell of claim 71, said cell containing a membrane bound, constitutively active transcription factor;
b) stimulating activity of a protease by the compound to release the transcription factor from the membrane and therefore allowing the transcription factor to translocate to the nucleus;
c) measuring expression of a gene under promotional control of the membrane bound transcription factor; and
d) determining the effect of the compound on the amount of gene expression.

73. A method comprising the steps:
a) contacting a compound and an extracellular ligand with a cell of claim 71, said cell containing a membrane bound, constitutively active transcription factor;
b) stimulating activity of a protease by either the compound or the ligand to release the transcription factor from the membrane and therefore allowing the transcription factor to translocate to the nucleus;
c) measuring expression of a gene under promotional control of the membrane bound transcription factor; and
d) determining the effect of the compound on the amount of gene expression.

74. A compound discovered using the method of claim 27.

75. A pharmaceutical composition comprising a compound of claim 74.

76. A compound discovered using the method of claim 39.

77. A pharmaceutical composition comprising a compound of claim 76.

78. A compound discovered using the method of claim 67.

79. A pharmaceutical composition comprising a compound of claim 78.

80. A compound discovered using the method of claim 68.

81. A pharmaceutical composition comprising a compound of claim 81.

82. A compound discovered using the method of claim 72.

83. A pharmaceutical composition comprising a compound of claim 83.

84. A compound discovered using the method of claim 73.

85. A pharmaceutical composition comprising a compound of claim 84.
